SEQUENCE LISTING (2 sequences)

SEQ ID NO: 1 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 6 aa, Homo sapiens
VDPDNE

(19) United States
(12) Patent Application Publication
(10) Pub. No.: US 2026/0219283 A1
(43) Pub. Date: Jul. 30, 2026

(54) A DUPLEX ELECTROCHEMILUMINESCENCE IMMUNOASSAY FOR TOTAL A-SYNUCLEIN AND PS129-A-SYNUCLEIN

(71) Applicant: The Regents of the University of California, Oakland, CA (US)

(72) Inventors: Gal Bitan, Encino, CA (US); Suman Dutta, Los Angeles, CA (US); Karl Biggs, San Pedro, CA (US); Ibrar Siddique, Los Angeles, CA (US)

(73) Assignee: The Regents of the University of California, Oakland, CA (US)

(21) Appl. No.: 19/146,842

(22) PCT Filed: Jan. 30, 2024
(86) PCT No.: PCT/US2024/013520

§ 371 Date: Jul. 9, 2025

Related U.S. Application Data
(60) Provisional application No. 63/482,173, filed on Jan. 30, 2023.

(51) Int. Cl. G01N 33/68; C09K 11/06; G01N 33/543; G01N 33/58

(52) U.S. Cl. CPC G01N 33/6896; C09K 11/06; G01N 33/5438; G01N 33/582; G01N 2800/28

(57) ABSTRACT

Synucleinopathies are a group of neurodegenerative diseases including Parkinson's disease (PD), dementia with Lewy bodies (DLB), and multiple system atrophy (MSA). These diseases are characterized by the aggregation and deposition of α-synuclein (α-syn) in Lewy bodies (LBs) in PD and DLB or as glial cytoplasmic inclusions in MSA. In healthy brains, only ~4% of α-syn is phosphorylated at Ser129 (pS129-α-syn), whereas >90% pS129-α-syn may be found in LBs. Embodiments of the invention include a duplex assay for both total α-synuclein and pS129-α-synuclein, which allows measuring both analytes in the same sample, leading to substantial saving in sample volume. The assays can be widely used in methods for detecting pS129-α-syn in biomedical studies including when only a limited volume of sample is available and high sensitivity is required, offering new opportunities for diagnostic biomarkers, monitoring disease progression, and quantifying outcome measures in clinical trials.

### CROSS REFERENCE TO RELATED APPLICATIONS

This application claims the benefit under 35 U.S.C. Section 119 (e) of co-pending and commonly-assigned U.S. Provisional Patent Application Ser. No. 63/482,173, filed on Jan. 30, 2023 and entitled “AN ELECTROCHEMILUMINESCENCE ELISA FOR PS129-A-SYNUCLEIN” which application is incorporated by reference herein.

### TECHNICAL FIELD

The invention relates to methods and systems for examining α-Synuclein.

### BACKGROUND OF THE INVENTION

α-Synuclein self-assembly into neurotoxic oligomers and aggregates underlie neurodegenerative diseases called synucleinopathies, including Parkinson's disease (PD), dementia with Lewy bodies, and multiple system atrophy. Neurotoxic assemblies of α-synuclein are also found in the central nervous system in PD dementia (PDD), the Lewy-body variant of Alzheimer's disease (LB-AD), traumatic brain injury, and spinal cord injury. A form of α-synuclein phosphorylated at serine 129 (pS129-α-synuclein) is highly elevated in these pathologic conditions and therefore could be useful as a marker of disease, disease progression, and/or treatment impact. The level of pS129-α-syn is only ~4% in normal brain but it increases to ~90% in Lewy bodies and is elevated in other synucleinopathies (see, e.g. Oueslati, A. (2016) J Parkinsons Dis 6, 39-51).

However, although detecting pS129-α-synuclein immunohistochemically is straightforward, measuring it biochemically in tissue extracts or bodily fluids, especially in cases in which high sensitivity is required, has been difficult. This difficulty has impeded a wide use of pS129-α-synuclein as a biomarker.

A number of reasons have created the difficulty in developing a reproducible, high-sensitivity biochemical assay for pS129-α-synuclein including the variability in the sensitivity and specificity of anti-pS129-α-synuclein antibodies and the challenge of obtaining pure pS129-α-synuclein for generating a standard curve. In studies published to date measuring pS129-α-syn biochemically, a variety of antibodies with variable specificity have been used for detection with highly variable sensitivity and specificity (see, e.g. Delic et al., J. Comp. Neurol. 526, 1978-1990). For generating a standard curve, previous studies used recombinant α-syn phosphorylated by casein kinase II (See, e.g. Anderson, et al., (2006) J. Biol. Chem. 281, 29739-29752; Foulds et. al. (2011) FASEB J 25, 4127-4137; Wang et al., (2012) Sci Transl Med 4; and Majbour et al., (2016) Mol. Neurodegener. 11, 7) or polo-like kinase 2 (see, e.g. Landeck et al., (2016) Mol. Neurodegener. 11, 61), raising concerns regarding the completion of the phosphorylation at serine 129 and potential phosphorylation at other sites, which might cross-react with the antibodies used in the assay (see, e.g. Waxman et al., J Neuropathol Exp Neurol 67, 402-416).

Conventional color-based ELISAs for pS129-α-synuclein are known in the art and commercially available. However, the reported limit of detection of such assays is 3.12 ng/ml, which is not sufficient for most biomarker studies, for example the measurement of this biomarker in brain-derived blood exosomes which require sensitivity 1000-times higher than the sensitivity of color-based ELISAs. In view of this, there is a need in the art for assays that can measure pS129-α-synuclein present in picogram per milliliter concentrations in biological samples.

### SUMMARY OF THE INVENTION

As noted above, there is a need for sensitive assays for pS129-α-synuclein, particularly in the context of biomarker development for Parkinson's disease (PD) and other synucleinopathies. In view of this, we have developed a technology that is capable of measuring picogram per milliliter concentrations of pS129-α-synuclein in biological samples such as brain-derived blood exosomes. To reach this high sensitivity, we have designed a new assay based upon electrochemiluminescence (ECL) ELISA technology. While the specification disclosure focuses on electrochemiluminescence ELISA assays, other embodiments of the invention include ELISA assays designed to detect pS129-α-synuclein based on color, fluorescence, chemiluminescence and the like.

Embodiments of the invention include duplex assays designed to measure both phosphorylated and unphosphorylated α-synuclein in the same sample, leading to substantial saving in biological sample volumes used for the assays. Initial attempts to develop this assay showed that the signal of pS129-α-synuclein was reduced by several orders of magnitude compared to the singlex assay, making it impractical. To overcome this issue, the concentrations of the capture antibodies were tested systematically leading to the discovery that the capture antibodies—an antibody against total α-synuclein provided as part of the U-PLEX kit from Meso Scale Discovery (MSD), and anti-pS129-α-synuclein antibody EP1536Y needed to be added to the plate at a selected concentration ratio in order to compensate for the apparent higher affinity of the antibody against total α-synuclein. In addition, in certain embodiments of the invention, the incubation time with recombinant pS129-α-synuclein was extended from 2 hours at room temperature to 12 hours at 4° C. This unexpectedly led to an order-of-magnitude improvement in the signal for pS129-α-synuclein, thereby optimizing the duplex assays.

The invention disclosed herein has a number of embodiments. One embodiment of the invention is a method for measuring pS129-α-synuclein levels in a sample, for example a biological sample obtained from an individual diagnosed with or suspected of having a synucleinopathy such as Parkinson's disease (e.g. a biological sample selected to comprise brain-derived blood exosomes from the individual). Embodiments of this method typically comprise attaching a pS129-α-synuclein capture antibody to a matrix such as a bead or wall of a vessel in which the pS129-α-synuclein sample is disposed, and then introducing a pS129-α-synuclein sample into the vessel so that pS129-α-synuclein binds the capture antibody. In this method a pS129-α-synuclein detection antibody is introduced into the container so that the pS129-α-synuclein detection antibody binds pS129-α-synuclein, with this detection antibody comprising a label such as an electrochemiluminescent label. Electrochemiluminescent methods then comprise applying electricity to an electrode material within the container so that light is generated by the electrochemiluminescent label; measuring light emitted by the electrochemiluminescent label; and then correlating the measured light to a concentration of pS129-α-synuclein in the container. The labels in this assay allow for ultra-sensitive detection of pS129-α-synuclein. Embodiments of the invention disclosed herein include immunoassays that can measure pS129-α-synuclein present in biological samples at levels below 10 μg/mL (e.g. between 1-9 μg/mL). In certain electrochemiluminescent embodiments of the invention, electrodes are disposed in an assay container such as the bottom of SINGLE/MULTI-ARRAY and/or SINGLE/MULTI-SPOT microplates to allow for easy attachment of biological reagents.

The methods of the invention typically include the step of observing a pS129-α-synuclein polypeptide standard/control, for example to generate a standard curve, wherein the pS129-α-synuclein standard/control is selected/formed to be in a relatively unaggregatable form (e.g. selected to be one that remains in an unaggregated form following at least one month of storage at −80° C.). In illustrative embodiments of the invention, a pS129-α-synuclein capture antibody is attached to a vessel surface, and a pS129-α-synuclein detection antibody is sulfonated for producing an electrochemiluminescent signal. In working embodiments of the invention, we used the monoclonal antibody MJFR-1, which is specific for human α-synuclein, and the monoclonal antibody EP1536Y. We tested this pair of antibodies in both configurations—biotinylated MJFR-1 for capture and sulfo-tagged EP1536Y for detection and vice versa. We further used a commercial, semi-synthetic pS129-α-synuclein as a standard/control target polypeptide to be assayed to generate a standard curve. In parallel we used the same concentrations of unphosphorylated α-synuclein as a negative control to test the specificity of the assay for pS129-α-synuclein. The assay quantifies pS129-α-synuclein with a sensitivity limit in the single pg/mL range.

Embodiments of the invention include duplex assays for measuring amounts of total α-synuclein and amounts of pS129-α-synuclein within a single sample. Typically these methods comprise combining the sample with a α-synuclein capture antibody and a α-synuclein detection antibody coupled to a detectable label; as well as a pS129-α-synuclein capture antibody and α-synuclein detection antibody coupled to a detectable label. Certain of these embodiments of the invention use two separate capture antibodies and the same detection antibody. In certain methods of the invention, the α-synuclein capture antibody is selected to exhibit an affinity for α-synuclein greater than an affinity that the pS129-α-synuclein capture antibody exhibits for pS129-α-synuclein; and amounts of the pS129-α-synuclein capture antibody and the amounts of the α-synuclein capture antibody are selected to form a concentration ratio (e.g., at least 1:8, 1:10, 1:20 or 1:30) that takes into account the different antibody affinities for α-synuclein and pS129-α-synuclein in order to optimize assay sensitivity. Such methods include the steps of allowing the α-synuclein capture antibody and the α-synuclein detection antibody to bind α-synuclein, and allowing the pS129-α-synuclein capture antibody and the pS129-α-synuclein detection antibody to bind pS129-α-synuclein and then observing the presence of the detectable labels, followed by correlating the observed detectable labels to concentrations of α-synuclein and pS129-α-synuclein in the sample such that amounts of total-synuclein and amounts of pS129-α-synuclein in the single sample are measured. In certain embodiments of the invention, the sample (e.g. human CSF, scrum, plasma, or saliva) volume is less than 2, 5, 10, 25, or 50 microliters. In embodiments of the invention, the method can quantify amounts of pS129-α-synuclein in the sample in concentrations from about 1 to about 9 μg/mL.

In some embodiments of the invention, these duplex assay methods include observing pS129-α-synuclein capture antibody binding to a recombinant pS129-α-synuclein polypeptide standard. In certain methods of the invention, the pS129-α-synuclein standard is selected to be in an unaggregated form. In some embodiments of the invention, the pS129-α-synuclein capture antibody and the recombinant pS129-α-synuclein polypeptide are incubated for at least 3, 6 or 12 hours. In some embodiments of the invention, the pS129-α-synuclein capture antibody and the recombinant pS129-α-synuclein polypeptide are incubated at 4° C. Typically, the methods can comprise at least one step wherein α-synuclein detection antibody not bound to α-synuclein and/or pS129-α-synuclein detection antibodies not bound to pS129-α-synuclein are removed from antibodies bound to phorphorylated or unphosphorylated α-synuclein polypeptide in the assay by a wash process or the like.

The duplex assay methods of the invention can be used to observe amounts of total α-synuclein and amounts of pS129-α-synuclein within a single sample in a wide variety of biological samples. For example, in some embodiments of the invention, the sample is selectively obtained from an individual diagnosed with or suspected of having a synucleinopathy (e.g. Parkinson's disease, lewy body dementia, multiple system atrophy or the like). In other embodiments of the invention, the sample is selectively obtained from an individual who has suffered a traumatic brain injury or a spinal cord injury. In related embodiments, the sample is selectively obtained from an individual being treated for a synucleinopathy; or an individual being treated for a traumatic brain injury or a spinal cord injury. Optionally, the method measures levels of α-synuclein and pS129-α-synuclein present in brain-derived blood exosomes.

In some embodiments of the invention, the duplex assay methods include an electrochemiluminescent process for measuring α-synuclein and pS129-α-synuclein in a sample. Typically these methods can include attaching a pS129-α-synuclein capture antibody to a matrix within a container, wherein the container comprises an electrode; introducing a pS129-α-synuclein sample into the container so that pS129-α-synuclein binds the capture antibody; introducing a pS129-α-synuclein detection antibody to the container so that the pS129-α-synuclein detection antibody binds pS129-α-synuclein, wherein the detection antibody comprises an electrochemiluminescent label; applying electricity to the electrode so that light is generated by the electrochemiluminescent label; measuring light emitted by the electrochemiluminescent label; and correlating the measured light to a concentration of pS129-α-synuclein in the container.

Embodiments of the invention include duplex assay systems for detecting amounts of total α-synuclein and amounts of pS129-α-synuclein in a single sample. Typically such systems include a pS129-α-synuclein polypeptide standard, wherein the pS129-α-synuclein standard is in an unaggregated form; a pS129-α-synuclein capture antibody and a pS129-α-synuclein detection antibody coupled to an observable label; and a α-synuclein capture antibody and a α-synuclein detection antibody coupled to an observable label. In such systems, the α-synuclein capture antibody exhibits an affinity for α-synuclein greater than an affinity that the pS129-α-synuclein capture antibody exhibits for pS129-α-synuclein; and amounts of the pS129-α-synuclein capture antibody and the amounts of the α-synuclein capture antibody are selected to form a concentration ratio that compensates for the different antibody affinities in order to optimize assay sensitivity. Typically for example, the concentration ratio is at least 1:8, 1:20 or 1:30

In certain embodiments of the invention, the duplex assay system comprises a defined amount of a pS129-α-synuclein standard polypeptide useful to generate a standard curve in an electrochemiluminescent ELISA assay, and/or the pS129-α-synuclein standard/control is selected to remain in an unaggregated form following at least one month of storage at −80° C. In some embodiments, the pS129-α-synuclein polypeptide comprises a semi-synthetic pS129-α-synuclein polypeptide that has been synthesized incorporating a phosphoserine residues at position 129. In some embodiments of the invention, the system comprises unphosphorylated S129-α-synuclein polypeptide useful as a negative control. Optionally, the system quantifies amounts of pS129-α-synuclein in a sample obtained from an individual in concentrations ranging from about 1 to about 9 μg/mL in an electrochemiluminescent ELISA assay. In certain embodiments, the duplex assay systems are designed so that the pS129-α-synuclein capture antibody and the recombinant pS129-α-synuclein polypeptide can be incubated together for at least 3, 6 or 12 hours; and/or the pS129-α-synuclein capture antibody and the recombinant pS129-α-synuclein polypeptide can be incubated at 4° C. In certain embodiments of the invention, the duplex assay system comprises a container for performing an electrochemiluminescent ELISA assay (e.g. a container comprising an electrode material).

Embodiments of the invention further include duplex assay kits comprising reagents (e.g. anti-pS129-α-synuclein antibodies, pS129-α-synuclein polypeptide controls and the like) for this assay, systems and kits that are useful in research laboratories, clinical laboratories, and the pharmaceutical industry. The high sensitivity and specificity of the assays disclosed herein and the relative ease by which they can be created and reproduced make them a highly attractive tool for research, clinical trials, and routine clinical use. Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1. Standard curves for pS129-α-syn (red) and total α-syn standards (blue) using different antibody configurations. A) Biotinylated EP1536Y used for capture and Sulfo-tagged MSD's anti-human-α-synuclein antibody for detection. B) Biotinylated MSD's anti-human-α-synuclein antibody used for capture and Sulfo-tagged EP1536Y for detection. C) Biotinylated mAb MJFR1 used for capture and Sulfo-tagged EP1536Y for detection. Representative standard curves (Mean±SD of two technical replicates) of at least five independent experiments are shown.

FIG. 2. Measurement of pS129 α-syn in human and mouse samples. All measurements were performed using biotinylated EP1536Y for capture and MSD's Sulfo-tagged anti-human-α-synuclein antibody for detection. A) Measurement of pS129-α-syn in human CSF, serum, plasma, and saliva. HC—healthy control, PD—Parkinson's disease, DLB—dementia with Lewy bodies, MSA—multiple system atrophy, LLoD—lower limit of detection, LLoQ—lower limit of quantitation. B) Comparison of pS129 α-syn concentrations in brain extracts from wild-type and MSA model mice. The data are shown as mean±SD.

FIG. 3. Dilution linearity and spike recovery. A-C) Dilution linearity within a 2-fold dilution series in A) human CSF from a patient with DLB, B) pooled human serum, and C) extracts of PLP-α-syn mouse brain. D-F) Spike recovery rates after addition of 250, 500, or 1000 μg/mL semi-synthetic pS129-α-syn to D) human CSF from a patient with DLB, E) pooled human serum, and F) extracts of PLP-α-syn mouse brain. The data are shown as mean±SD.

FIG. 4. Mass spectrometry analysis of the pS129 α-syn standard. An ESI mass-spectrum of pS129-α-syn. The inset shows the deconvoluted spectrum corresponding to a single protein species with the correct mass of pS129-α-syn.

FIG. 5. SDS-PAGE and western-blot analysis of the pS129-α-syn standard prepared using non-optimized conditions. Different quantities of the semi-synthetic pS129-α-syn standard were fractionated and unphosphorylated α-syn was used as a control. A) Coomassie Blue staining shows the presence of oligomers, presumably tetramers, and larger aggregates of pS129-α-syn but not of unphosphorylated α-syn. B) Higher-sensitivity visualization of the same gel by silver staining. C) Western blot analysis probed with the anti-α-syn antibody MJFR1. D) Western blot analysis probed with the anti-pS129-α-syn antibody EP1536Y. The gel migration of molecular weight markers is shown on the left in each panel.

FIG. 6. SDS-PAGE and western-blot analysis of the pS129-α-syn standard prepared using optimized conditions. Different quantities of the semi-synthetic pS129-α-syn standard were fractionated and unphosphorylated α-syn was used as a control. A) Coomassie Blue staining shows an absence of oligomers of pS129 α-syn but not of wild-type α-syn. B) Higher-sensitivity visualization of the same gel by silver staining shows minor bands of a putative dimer in the 0.5- and 1.0-μg pS129-α-syn lanes. Minor degradation products are also observed under the monomer band. Bands between 50-60 kDa likely are keratin contamination and are not related to the analyzed proteins. C) Western blot analysis probed with the anti-α-syn antibody MJFR1 showing minor putative dimer bands in the 0.5- and 1.0-μg pS129-α-syn lanes. D) Western blot analysis probed with the anti-pS129-α-syn antibody EP1536Y. The gel migration of molecular weight markers is shown on the left in each panel.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

An ever-expanding field of applications exists for rapid, highly specific, sensitive, and accurate methods of detecting and quantifying chemical, biochemical, and biological substances, including biomolecules associated with various pathologies (e.g. synucleinopathies) that can be found in biological samples. Because the amount of a particular biomolecule of interest such as polypeptides associated with a pathological condition (e.g. pS129-α-synuclein polypeptides) in a typical biological sample is often quite small, analytical biochemists are engaged in ongoing efforts to improve assay performance characteristics such as sensitivity.

One approach to improving assay sensitivity has involved amplifying the signal produced by a detectable label associated with the analyte of interest. In this regard, luminescent labels are of interest. Such labels are known which can be made to luminesce through photoluminescent, chemiluminescent, or electrochemiluminescent techniques. “Photoluminescence” is the process whereby a material luminesces subsequent to the absorption by that material of light (alternatively termed electromagnetic radiation or emr). Fluorescence and phosphorescence are two different types of photoluminescence. “Chemiluminescence” processes entail the creation of the luminescent species by a chemical reaction. “Electrochemiluminescence” is the process whereby a species luminesces upon the exposure of that species to electrochemical energy in an appropriate surrounding chemical environment.

The signal in each of these three luminescent techniques is capable of very effective amplification (i.e., high gain) through the use of known instruments (e.g., a photomultiplier tube or pmt) which can respond on an individual photon by photon basis. However, the manner in which the luminescent species is generated differs greatly among and between photoluminescent, chemiluminescent, and electrochemiluminescent processes. Moreover, these mechanistic differences account for the substantial advantages as a bioanalytical tool that electrochemiluminescence enjoys vis a vis photoluminescence and chemiluminescence. Some of the advantages possible with electrochemiluminescence include: (1) simpler, less expensive instrumentation; (2) stable, nonhazardous labels; and (3) increased assay performance characteristics such as lower detection limits, higher signal to noise ratios, and lower background levels.

In the context of certain bioanalytical chemistry measurement techniques, electrochemiluminescence can provide advantages over both photoluminescence and chemiluminescence. U.S. Pat. Nos. 5,147,806, 5,068,808, 5,061,445, 5,296,191, 5,247,243, 5,221,605, 5,238,808 and 5,310,687, the disclosures of which are incorporated herein by reference, detail certain methods, apparatuses, chemical moieties, inventions, and associated advantages of ECL. Another ECL system is described in a paper by Yang et al., Bio/Technology, 12, pp. 193-194 (February 1994). See also a paper by Massey, Biomedical Products, October 1992 as well as U.S. Pat. Nos. 5,235,808 and 5,310,687, the contents of these papers and patents being incorporated herein by reference.

The ECL concept has been successfully adapted to a number of platforms such as an electrode system within a microfluidic channel using one- or two-compartment cell containing one, two, or three electrodes in early 2000's. See, e.g., Wei Zhan, Julio Alvarez and Richard M. Crooks, “Electrochemical Sensing in Microfluidic System Using Electrogenerated Chemiluminescence as a Photonic Reporter of Redox Reactions”, J. Am. Chem. Soc., 124 (2002) 13265-13270; Wei Zhan, Julio Alvarez and Richard M. Crooks, “A Two-Channel Microfluidic Sensor That Uses Anodic Electrogenerated Chemiluminescence as a Photonic Reporter of Cathodic Redox Reactions”, Anal. Chem., 75 (2003) 313-318.; Wei Zhan, Julio Alvarez and Richard M. Crooks, “A Multi-Channel Microfluidic Sensor That Detects Anodic Redox Reactions Indirectly Using Anodic Electrogenerated Chemiluminescence”, Anal. Chem., 75 (2003) 1233-1238. Traditionally, an electrochemical cell is composed of electrodes such as a working electrode, a counter electrode, and a reference electrode. The reference electrode usually is in its equilibrium state and has a high resistance, and has its intrinsic electrode potential. It is usually connected to the ground. If the reference electrode were eliminated, the resulting two electrode system can work while there is no “electrode potential,” only a voltage between two electrodes.

As noted above, technical challenges in developing a reproducible, high-sensitivity biochemical assay for pS129-α-synuclein include the variability in the sensitivity and specificity of anti-pS129-α-synuclein antibodies as well as the challenge of obtaining pure pS129-α-synuclein for generating a standard curve. In studies measuring pS129-α-syn biochemically, a variety of antibodies with variable specificity have been used for detection with highly variable sensitivity and specificity. For generating a standard curve, previous studies used recombinant α-syn phosphorylated by casein kinase II or polo-like kinase 2, raising concerns regarding the completion of the phosphorylation at serine 129 and potential phosphorylation at other sites, which can cross-react with the antibodies used in the assay. A recent study has compared several commercial anti-pS129-α-synuclein antibodies and found the rabbit monoclonal antibody EP1536Y to be superior to all others (see, e.g. Delic et al., (2018) Sensitivity and specificity of phospho-Ser129 alpha-synuclein monoclonal antibodies. J. Comp. Neurol. 526, 1978-1990). For generation of a standard curve, semi-synthetic pS129-α-synuclein (see, e.g. Fauvet et al., (2016) Methods Mol. Biol. 1345, 3-20) has become commercially available by Proteos, Inc. Utilizing such newly available materials and building upon prior antibody studies, the inventors discovered antibodies having certain characteristics that make them well suited for ECL assays disclosed herein.

In developing the pS129-α-synuclein ELISA assays disclosed herein, we unexpectedly discovered that a deterioration of the signal of the pS129-α-syn standard used in these assays was occurring within 1-2 weeks of storage, prompting a detailed investigation to identify the possible causes of this issue and potential solutions. In view of the observed pS129-α-syn standard signal deterioration during the storage of the diluted and aliquoted protein standard, we tested whether it might have lost the phosphate group on Ser129. Examination of the protein by ESI-MS revealed that it had the mass with the expected phosphorylation, ruling out this option. We hypothesized next that the loss of signal could be due to oligomerization or aggregation of the protein during the preparation process, creating seeds that promoted further rapid aggregation, even when the protein is stored at −80° C. Indeed, by modifying the methods to ensure that the pS129-α-synuclein standard/control is selected to be in an unaggregated form (e.g. so that trifluoroacetic acid used to prepare the pS129-α-syn standard was removed completely from solution), such method steps prevented seed formation and allowed the pS129-α-synuclein standard/control protein to remain unaggregated for prolonged storage. Under these conditions, weekly repeated testing of the standard curve and positive control samples (MSA mouse-brain extract) yielded consistent data over one month without apparent signal loss.

As discussed in detail below, the invention disclosed herein has a number of embodiments of immunoassays that can detect and measure pS129-α-synuclein in a sample using ELISA methods to detect biomolecules via colorimetric imaging, fluorescent imaging, chemiluminescent imaging and the like. Embodiments of the invention include duplex assays designed to measure both phosphorylated and unphosphorylated α-synuclein the same sample, leading to substantial saving in biological sample volumes used for the assays. Initial attempts to develop this assay showed that the signal of pS129-α-synuclein was reduced by several orders of magnitude compared to the singlex assay, making it impractical. To overcome this issue, the concentrations of the capture antibodies were tested systematically leading to the discovery that the capture antibodies—an antibody against total α-synuclein provided as part of the U-PLEX kit from Meso Scale Discovery (MSD), and anti-pS129-α-synuclein antibody EP1536Y needed to be added to the plate at a 1:30 concentration ratio in order to compensate for the apparent higher affinity of the antibody against total α-synuclein. Of the 10-linker kit sold by MSD, Linker 1 was used for attachment of antibody EP1536Y to the plate, whereas Linker 10 was used for attachment of the antibody against total α-synuclein. In addition, in certain embodiments of the invention, the incubation time with recombinant pS129-α-synuclein was extended from 2 hours at room temperature to 12 hours at 4° C. This led to an order-of-magnitude improvement in the signal for pS129-α-synuclein, allowing successful use of the duplex assay.

Embodiments of the invention include duplex assay systems for detecting amounts of total α-synuclein and amounts of pS129-α-synuclein in a single sample. Typically such systems include a pS129-α-synuclein polypeptide standard, wherein the pS129-α-synuclein standard is in an unaggregated form; a pS129-α-synuclein capture antibody and a pS129-α-synuclein detection antibody coupled to an observable label; and α-synuclein capture antibody and a α-synuclein detection antibody coupled to an observable label. In such systems, the α-synuclein capture antibody exhibits an affinity for α-synuclein greater than an affinity that the pS129-α-synuclein capture antibody exhibits for pS129-α-synuclein; and amounts of the pS129-α-synuclein capture antibody and the amounts of the α-synuclein capture antibody are selected to form a concentration ratio that compensates for the different affinities in order to optimize assay sensitivity. Typically for example, the concentration ratio is at least 1:20, 1:30 or 1:40.

As noted above, embodiments of the invention include for example, electrochemiluminescent methods for measuring pS129-α-synuclein in a sample. Such methods can comprise attaching a pS129-α-synuclein capture antibody to a matrix such as a wall of an ELISA plate (or a similar container/vessel), a microbead or the like. In this method, the assay is performed in a vessel/container that comprises one or more materials that function as an electrode (e.g. one or more high binding carbon electrodes). This methodology includes introducing a pS129-α-synuclein sample into the container so that pS129-α-synuclein binds the capture antibody, followed by introducing a pS129-α-synuclein detection antibody to the container so that the pS129-α-synuclein detection antibody binds pS129-α-synuclein. Typically in these methods, the detection antibody comprises a label such as an electrochemiluminescent label adapted for use in the specific assay. In such methods of measuring pS129-α-synuclein in a sample, the method includes observing a pS129-α-synuclein polypeptide standard/control (e.g. of a defined concentration), wherein the pS129-α-synuclein standard/control is selected to be in an unaggregated form. Typically in these methods, the electrode can be utilized (e.g. by applying electricity to the electrode) so that light is generated by the electrochemiluminescent label. These methods then comprise measuring light emitted by the electrochemiluminescent label; and correlating the measured light to a concentration of pS129-α-synuclein in the container. Embodiments of the invention include additional methodological steps commonly performed in ECL assays, for example at least one step wherein pS129-α-synuclein detection antibodies not bound to pS129-α-synuclein are removed from the container by a wash process. As discussed below, in certain embodiments of the invention, the method can measure/quantify pS129-α-synuclein in the sample in concentrations from about 1 to about 9 μg/mL.

The methods disclosed herein can measure/quantify levels of pS129-α-synuclein present in a variety of samples. For example, in illustrative embodiments of the invention, the method is adapted to measure levels of pS129-α-synuclein present in brain-derived blood exosomes. Typically in these methods, the brain-derived blood exosomes (e.g. those derived from serum or plasma samples) are subjected to at least one purification step prior to the immunoassay (e.g. one comprising an exosome precipitation step, a centrifugation step, an enrichment step, an incubation step or the like). In certain embodiments of the invention, the pS129-α-synuclein sample is selectively obtained from an individual diagnosed with or suspected of having a synucleinopathy (e.g. Parkinson's disease, lewy body dementia, multiple system atrophy or the like); and/or an individual who has suffered a traumatic brain injury or a spinal cord injury; and/or an individual being treated for a synucleinopathy; and/or an individual being treated for a traumatic brain injury or a spinal cord injury.

The invention disclosed herein is based in part upon the discovery that certain antibodies that bind pS129-α-synuclein exhibit a specificity that allows them to function in electrochemiluminescent methods that are sensitive enough to measure/quantify pS129-α-synuclein in samples in concentrations from about 1 to about 9 μg/mL. In particular, it is known in the art that phosphospecific antibodies often cross-react with non-specific antigens, a phenomenon which creates problems, especially when using such antibodies to assay phosphoproteins such as pS129-α-synuclein, particularly when the assays seek to measure very small amounts of the phosphoprotein (e.g. levels in the single picogram per milliliter range). In this context, the inventors unexpectedly discovered that certain well known and commercially available antibodies to pS129-α-synuclein exhibit a sensitivity that allows them to be used to design electrochemiluminescent methods that are sensitive enough to measure/quantify pS129-q-synuclein in samples in concentrations from about 1 to about 9 μg/mL. These antibodies include the antibody EP1536Y (see, e.g. Delic et al., J. Comp. Neurol. 2018 526 (12): 1978-1990). The antibody EP1536Y is commercially available and sold, for example, by ABCAM (“Recombinant Anti-Alpha-synuclein (phospho S129) antibody [EP1536Y] (ab51253)”). These antibodies also include the antibody MJRFR1 (see, e.g. Delic et al., J. Comp. Neurol. 2018 526 (12): 1978-1990). The antibody MJRFR1 is commercially available and sold, for example, by ABCAM (“Recombinant Anti-Alpha-synuclein (phospho S129) antibody [MJRFR1] (ab138501)”).

In certain embodiments of the invention, the capture antibody or the detection antibody is selected to bind a pS129-α-synuclein epitope bound by antibody MJFR-1 or a pS129-α-synuclein epitope bound by antibody EP1536Y. In some embodiments of the invention, the pS129-α-synuclein epitope comprises the amino acid sequence VDPDNE (SEQ ID NO: 2). In some embodiments of the invention, the detection antibody exhibits a sensitivity and/or specificity for pS129-α-synuclein epitope that is within 1-2 orders of magnitude of the sensitivity and/or specificity of the antibody EP1536Y for pS129-α-synuclein epitope. In this context, methods for determining the sensitivity and/or specificity of such antibodies are well known in the art (see, e.g., Bordeaux et al., Biotechniques. 2010 March; 48 (3): 197-209; Holmseth et al., J Histochem Cytochem. 2012 March; 60 (3): 174-187; Achrya et al., F1000Research 2017, 6:851; and chapter 63 of Cell Biology (Third Edition) A Laboratory Handbook Volume 1, 2006, Pages 527-532).

Embodiments of the invention include systems for detecting pS129-α-synuclein. Typically such systems comprise a pS129-α-synuclein capture antibody and a pS129-α-synuclein detection antibody coupled to a detectable marker. In certain embodiments of the invention, the detection antibody comprises an electrochemiluminescent label; and the combination of the pS129-α-synuclein capture antibody and the pS129-α-synuclein detection antibody can detect pS129-α-synuclein in concentration from about 1 to about 9 μg/mL in an electrochemiluminescent ELISA assay. In certain embodiments of the invention, the system comprises a defined amount of a pS129-α-synuclein polypeptide useful to generate a standard curve in the electrochemiluminescent ELISA assay. Typically, this pS129-α-synuclein polypeptide comprises a semi-synthetic pS129-α-synuclein polypeptide that has been synthesized incorporating a phosphoserine residue at position 129. In certain embodiments of the invention, the system comprises a defined amount of unphosphorylated S129-α-synuclein polypeptide useful as a negative control.

In some system embodiments of the invention the capture antibody or the detection antibody is selected to bind a pS129-α-synuclein epitope bound by antibody MJFR-1 or a pS129-α-synuclein epitope bound by antibody EP1536Y. Optionally, the detection antibody exhibits a sensitivity and specificity within 1 or 2 orders of magnitude of the sensitivity and specificity of the antibody EP1536Y. In certain embodiments of the invention, the pS129-α-synuclein epitope bound by the detection or capture antibody comprises the amino acid sequence VDPDNE (SEQ ID NO: 2). In certain embodiments of the invention, the system comprises a container (optionally comprising a material that functions as an electrode) for performing the electrochemiluminescent ELISA assay. Embodiments of the invention further include kits comprising the one or more elements of the system for detecting pS129-α-synuclein disclosed herein.

The methods and materials disclosed herein can be adapted for use with a number of conventional ECL assay formats. For example, embodiments of the invention can include multi-well plate embodiments such as those disclosed in U.S. Patent Publication No. 20140299468. In some embodiments, the ECL system can further comprise a detector, for detecting ECL signals. In other embodiments, the ECL system further comprises a data management system for reporting and analyzing signal data from the detector. In an additional embodiment, the ECL system further comprises a fluidic handling system for labeling, washing, and supplying and dispose of buffer and sample solutions.

As disclosed herein, embodiments of the invention can be adapted for use with a wide variety of ECL assays known in the art such as those produced by MESO SCALE DIAGNOSTICS, LLC. For example, embodiments of the invention can use microparticles comprised of an electrically conductive material. In particular, as disclosed in U.S. Patent Publication No. 20160370359, it has been found that benefits are achieved in electrochemiluminescence reactions using microparticles comprised of an electrically conductive material having (a) one or more copies of an assay-ligand immobilized on its outer surface and (b) a plurality of electrochemiluminescent moieties immobilized on its outer surface. The micro-particles may have a coating thereupon which the assay-ligand and electrochemiluminescent moieties are immobilized. In another embodiment the conductive microparticles have a plurality of copies of an assay-ligand labeled with an electrochemiluminescent moiety immobilized on its outer surface.

In one embodiment of the invention, the use of microparticles as labels provides for improved signal in ECL assays by providing a scaffold for multiple ECL moieties. While the use of binding reagents comprising multiple ECL moieties is known, the signal enhancement obtainable by that approach is limited for the following reasons: i) the number of labels that can be attached to a binding reagent is limited by the surface area of the reagent; ii) multiple labeling of a binding reagent may lead to denaturation and/or inactivation of the reagent; iii) multiple labeling of a reagent may interfere with its ability to bind other reagents, e.g., by blocking the active site; and iv) multiple labeling of a binding reagent may lead to quenching of the luminescent excited state of one or more labels due to crowding of the labels on one, e.g., protein or nucleic acid. By providing a scaffold for ECL moieties, said scaffold not being involved in the binding event and said scaffold having a large surface area compared to the binding reagent.

In certain embodiments of the invention, the use of microparticles as a scaffold for multiple ECL moieties provides for reduced non-specific binding between the ECL labels and/or i) other entities present in either the sample (e.g., proteins, nucleic acid and the like); ii) the assay reagents (e.g., assay ligands); iii) the instrumentation/materials used to perform ECL assays (e.g., a solid support, an electrode, a cell, and the like). Reduced non-specific binding can advantageously improve the performance of assay measurements in several ways, for example: i) by decreasing background (non-specific) signals to improve sensitivity and/or dynamic range; ii) by reducing or eliminating the necessity for wash steps during the assay process (thus reducing the cost, time, and complexity of ECL assays and instrumentation); iii); by allowing a multiple of different assay ligands to be present in the same assay (or reaction) media without excessively interfering with each other or with other assay reagents or ECL instrument, and iv) by allowing more ECL moieties to be incorporated in an ECL label (without incurring undue nonspecific binding), thus increasing the number of photons emitted per binding event (which may improve sensitivity, dynamic range, and/or reduce the cost of complexity of light detectors used for ECL assays).

In an illustrative embodiment of the invention, the microparticles are comprised of a conductive material, typically a highly conductive material. The use of some conductive particles as scaffolds for ECL moieties can lead to additional enhancements in ECL when compared to certain non-conductive particles. Without being bound by theory, it is believed that this additional enhancement is due to the ability of the particle itself to conduct electrons from the working electrode so as to oxidize or reduce ECL moieties on its surface. In an especially illustrative embodiment of the invention, the particles are comprised of a very highly conductive material.

In another illustrative embodiment of the invention, the microparticles comprise a material capable of acting as a working electrode for inducing ECL from a certain ECL moiety and a particular ECL coreactant (i.e., the material is “ECL active”). It is possible to determine if a material is ECL active for a particular combination of ECL moiety and coreactant by testing whether a sample of the material, when used as a working electrode in an appropriate electrochemical cell under appropriate conditions, induces ECL.

Embodiments of the invention using microparticles in assays for an analyte-of-interest in a sample comprise the steps of (a) forming a composition comprising (i) a sample, (ii) a microparticle having one or more copies of a first assay-ligand immobilized on its surface and a plurality of ECL moieties immobilized on its surface (iii) a second assay-ligand immobilized on an electrode; (b) incubating the composition to form a complex; and (c) conducting an ECL measurement in the presence of ECL reactants. Said first and second assay-ligands may be the same or different. A complex is thus formed including (i) a microparticle having one or more copies of an assay-ligand immobilized on its surface, and a plurality of copies of an ECL moiety immobilized on its surface, and (ii) an assay-ligand immobilized on an electrode.

In an alternate embodiment, the assay-ligand is immobilized on a solid-phase support other than an electrode, said solid-phase support being capable of being collected at (or brought into contact with) an electrode. Assays of this alternate embodiment comprise the steps of (a) forming a composition comprising (i) a sample, (ii) a microparticle having one or more copies of a first assay-ligand immobilized on its surface and a plurality of ECL moieties immobilized on its surface (iii) a second assay-ligand immobilized on a solid phase support; (b) collecting said solid-phase support at (or bringing said solid phase support into contact with) an electrode; and (c) conducting an ECL measurement in the presence of ECL reactants. Said first and second assay-ligands may be the same or different. A complex is thus formed including (i) a microparticle having one or more copies of an assay-ligand immobilized on its surface, and a plurality of copies of an ECL moiety immobilized on its surface, and (ii) an assay-ligand immobilized on a solid-phase support. Suitable apparatus and solid-phase supports (e.g., magnetic beads) for carrying out assays according to this embodiment include those disclosed in PCT published application WO92/14139 and PCT published application WO90/05301.

As noted above, the methods and compositions used in embodiments of the invention can be adapted for use in a wide variety of ELISA formats. Such formats include sandwich assays and competitive binding assays (see, e.g., the following references, hereby incorporated by reference: Nonradioactive Labeling and Detection of Molecules, Kessler, C., ed., Springer-Verlag: Berlin 1992; The Immunoassay Handbook, Wild, D., ed., Stackton Press: New York 1994; and Keller, G. H.; Manak, M. M. DNA Probes, 2nd Ed., MacMillan Publishers Ltd.: London, 1993; Tietz Textbook of Clinical Chemistry 2nd Edition, Burtis et al. Ed., W.B. Saunders and Co.: Philadelphia, 1994).

Further illustrative methods, materials and applications of the invention are described in the following examples.

### EXAMPLES

### Example 1: Development of a Novel Electrochemiluminescence ELISA for Quantification of A-Synuclein Phosphorylated at Ser129 in Biological Samples

Synucleinopathies, including Parkinson's disease (PD), dementia with Lewy bodies (DLB), and multiple system atrophy (MSA) are characterized clinically by a chronic and progressive decline in motor, cognitive, behavioral, and/or autonomic functions. Deposits of fibrillar α-synuclein (α-syn) as Lewy bodies (LBs) and Lewy neurites in PD and DLB, or as glial cytoplasmic inclusions (GCIs) in MSA, are pathological hallmarks of these diseases.^{1,2} Differential diagnosis of synucleinopathies is difficult due to clinical symptoms overlap, especially in early disease stages.³ Phosphorylated forms of α-syn, particularly at Ser129 (pS¹²⁹-α-syn) are highly enriched in LBs and GCIs and are thought to be related to the disease process.^{4, 5} Therefore, if pS¹²⁹-α-syn can be measured in bodily fluids of patients it could serve as a sensitive biomarker for improving diagnosis accuracy, measuring disease progression, and assessing therapeutic outcomes.^{6, 7}

Currently, commercially available assays for quantifying pS¹²⁹-α-syn are limited. Cell-based ELISAs are available from Abexxa (Cambridge, United Kingdom), Creative Diagnostics (Shirley, NY) and Aviva Systems Biology (San Diego, CA), but based on the information provided by the companies, these assays are not optimized for detecting the recombinant protein and are assumed to be less reproducible than typical ELISA. Cell-based assays show intrinsically high variability, mainly due to the difficulty of plating the exact number of cells in each well, and variation in cell proliferation rate and responsivity. A typical sandwich ELISA is sold by MyBioSource⁸ yet the company's website disclaims: “cross reaction to other targets may potentially exist” and, “cross-reactivity could vary between sample type or species”. In addition, the sensitivity limit of the assay, 3.12 ng/ml, may not be compatible with low concentrations of pS¹²⁹-α-syn in human samples available in limited quantities.

Previously, several groups have reported assays for pS¹²⁹-α-syn, which varied widely in sensitivity, and used them to measure the phospho-protein in human cerebrospinal fluid (CSF),⁹⁻¹¹ plasma,¹¹⁻¹³ human cell culture lysates,¹² human and rat brain lysates,¹² and human erythrocytes.¹⁴ The assays included traditional ELISA^{6, 9, 13} and higher-sensitivity methods, such as Singulex Erennall, Luminex¹⁰ and AlphaLISA.¹² Recently, the Zhang group created a pS¹²⁹-α-syn electrochemiluminescence ELISA (ECLIA) using biotinylated anti-pS¹²⁹-α-syn (BioLegend, San Diego, CA) and anti-α-syn antibody clone 42 (BD Bioscience, CA) labelled with Sulfo-TAG. Applying this assay, they quantified the concentration of pS¹²⁹-α-syn in the membrane and cytosolic fractions of erythrocytes isolated from blood samples of healthy controls (HC) and patients with PD.¹⁴ Despite the importance of pS¹²⁹-α-syn as a potential biomarker for diagnosis and measuring progression of synucleinopathies, none of these assays has become mainstream.

Three main issues likely have prevented general use of the aforementioned assays. First, the specificity and reproducibility of the antibodies used varied and some of the antibodies were not readily available to other research groups. Second, in most of the studies published to date, the standard curve was generated using recombinant α-syn phosphorylated in vitro by casein kinase II^{4, 6, 9, 10} or polo-like kinase 2¹² raising concerns regarding the completion of the phosphorylation at Ser¹²⁹ and possible phosphorylation of other sites, which might cross-react with the antibodies used in the assay or otherwise affect their binding to pS¹²⁹-α-syn.¹⁵ Third, most studies published to date focused on demonstrating the suitability of their assays only for a specific sample type, e.g., CSF^{9, 10} but did not report testing of different antibody combinations, fine-tuning the protocol, or comparing the assay in different biofluids. To address these concerns, we have extensively researched publications on commercially available antibodies and used as a standard the semi-synthetic pS¹²⁹-α-syn first reported by the Lashuel group¹⁶ and made available by the Michael J. Fox Foundation via Proteos Inc. We have considered multiple factors in selecting the antibodies, tested several methodological approaches, and evaluated the suitability of our assay to quantify pS¹²⁹. α-syn in several commonly analyzed biological sample types. A comparable study was published recently by Cariulo et al. in which they characterized different antibody combinations of anti-α-syn and anti-pS¹²⁹ α-syn antibodies and measured total and pS¹²⁹ α-syn in CSF and plasma using a Singulex Erenna immunoassay.¹¹

The novel ECLIA we describe here for measurement of pS¹²⁹-α-syn detects pg/mL concentrations of pS¹²⁹-α-syn and has a wide dynamic linear range and low intra- and inter-assay variability. It is suitable for use in multiple types of biological samples and thus provides a platform for measuring pS¹²⁹-α-syn as a biomarker for a variety of clinical and research applications.

### Experimental Section

Materials. Semi-synthetic pS¹²⁹-α-syn was obtained from Proteos Inc. (Kalamazoo, MI). Anti-pS¹²⁹-α-syn monoclonal antibody EP1536Y (ab209422) and anti-α-synuclein monoclonal antibody MJFR1 (ab138501) were procured from Abcam (Cambridge, UK). Small-spot streptavidin-coated 96-wells ELISA plates (L45SA), assay diluent (R50AM), recombinant human α-syn calibrator (C01WK), biotinylated anti-total α-syn antibody, SULFO-TAG anti-human α-syn antibody, GOLD SULFO-TAG NHS-Ester conjugation pack (R31AA) and read buffer (R92TC) were obtained from Meso Scale Discovery (MSD, Rockville, MD). Phosphate-buffered saline (PBS), Tris-buffered saline (TBS), and biotin quantitation kit were from Thermo Fisher Scientific (Waltham, MA).

Biological samples. Pooled human serum and pooled human plasma was procured from Innovative Research (Novi, MI). Cerebrospinal fluid samples collected postmortem were obtained from the UCLA Division of Neuropathology (2 PD, 2 DLB, 3 MSA), UC Irvine Institute for Memory Impairments and Neurological Disorders (7 healthy controls), and Banner Sun Health Research Institute, Sun City, AZ (1 PD). Saliva was collected from healthy volunteers in our research group. Mouse brain extracts were from PLP-α-syn mice, a model of experimental MSA, or wild-type littermates and were obtained as described previously.¹⁷

Preparation of standard. The pS¹²⁹-α-syn standard was prepared according to the protocol published previously by Cariulo et al.¹¹ Briefly, lyophilized protein was weighed and dissolved in 100% trifluoroacetic acid (TFA) from Alfa Aesar (Haverhill, MA). One μl of TEA was added per 25 μg of protein. The TFA was evaporated completely in a fume hood for 1 h and the protein then was dissolved in TBS (50 mM Tris, 150 mM NaCl, pH 7.4) at a concentration of 20 μM. The solution was filtered through a 100-kDa cutoff filter from Pall (Show Low, AZ) and protein concentration was measured using a bicinchoninic acid (BCA) assay (Thermo Fisher Scientific, Waltham, MA). 1% (v/v) Tween-20 (Fisher BioReagents, Pittsburgh, PA) was added and the solution was aliquoted and stored at −80° C.

Biotinylation and sulfonation of antibodies. For biotinylation of antibody EP1536Y, 40 μL of a 0.90-mg/mL solution of the antibody were incubated with 1.61 μL of 11 mM Sulfo-NHS-Biotin (50-fold molar excess) on ice for 2 h according to the manufacturer's protocol (EZ-Link™ Micro Sulfo-NHS-LC-Biotinylation Kit, Thermo Fisher Scientific). Excess free biotin was removed using a 0.5-mL 7-kDa MWCO Zeba™ Spin Desalting Column (Thermo Fisher Scientific). Two hundred μL of ultra-pure water (purified using a milliQ system, Millipore, Burlington, MA) were added as a stacker volume for elution of the biotinylated EP1536Y antibody. Forty μL of 1.167-mg/mL solution of antibody MJFR1 were biotinylated using the same biotinylation kit. Biotinylation levels were evaluated using a fluorescence biotin quantitation kit (Thermo Fisher Scientific). For sulfonation, 40 μL of a 0.90-mg/mL of EP1536Y solution were incubated with SULFO-TAG NHS-Ester (MSD) according to the manufacturer's instructions. The protein concentration of all antibody conjugates was determined using a Pierce BCA assay kit (Thermo Fisher Scientific). The antibody solutions were aliquoted and stored at 4° C. until use.

Preparation of biological samples. CNS-originating extracellular vesicles (EVs) were isolated from the serum of healthy controls (HC), patients with PD, and patients with MSA as described previously.¹⁸ For analysis of pS¹²⁹-α-syn in the CSF of patients with different synucleinopathies and healthy controls, as well as in CNS-originating EVs isolated from human serum, 15 μg total protein, determined using a BCA assay, in 25 μL of Diluent 49 (MSD) were analyzed. Saliva samples obtained from healthy volunteers were centrifuged briefly at 10,000 g, the clear supernates were collected, and a final volume of 25 μL was used for analysis. Twenty-five μL of clear pooled human serum or plasma containing 1×Halt™ Protease and Phosphatase Inhibitor Cocktail (P/PI, Thermo Fisher Scientific) were loaded directly onto the wells. MSA or wild-type mouse brain lysates (5 μg total protein) were diluted in 25 μL Diluent 49 for ECLIA analysis.

ECLIA. The assay was developed using MSD gold 96-well small-spot streptavidin SECTOR ELISA plates. Capture and detection antibody concentrations were determined based on the information available on MSD's website for validation of human α-synuclein assay. We found that a concentration of 2 μg/mL antibody was sufficient for the capture antibody to saturate the wells of the streptavidin-coated plates. Biotinylated-antibody stock solutions were diluted in 1% (w/v) biotin-free bovine serum albumin (bf-BSA) in TBS containing 0.1% (v/v) Tween-20 (TBS-T) to reach a concentration of 2 μg/mL EP1536Y, MJRF1, or MSD's 1×anti-α-syn capture antibody. Dilutions of the semi-synthetic pS¹²⁹-α-syn, recombinant human α-syn standards, and biological samples were carried out using Diluent 49 (MSD), which also was used as a “zero” calibrator (blank). One hundred fifty microliters of 3% (w/v) bf-BSA in TBS-T were added to each well and the plates were incubated with shaking at 800 rpm at RT for 1 h. The blocking solution then was removed and 25 μL of 2 μg/mL or IX biotinylated capture antibodies were added to each well. The plates were incubated with shaking at 800 rpm at RT for 1 h and then washed three times with 150 μL TBS-T per well. Subsequently, 25 μL of SULFO-TAG detection antibody and 25 L of samples or standards were added to the wells and the plates were incubated further with shaking at 800 rpm at RT for 2 h. After washing three times with 150 μL TBS-T per well, 150 μL of 1×read buffer (MSD) were added to each well and the plates were read immediately using a Sector S600 reader (MSD). The data were analyzed using Discovery Workbench 4.0 software (MSD) and quantified with reference to freshly prepared standard curves. Spike recovery. To test spike recovery, human CSF, serum, and PLP-α-syn mouse brain lysates, or Diluent 49 as a control, were spiked with 250 μg/mL (low spike), 500 μg/mL (medium spike) or 1,000 μg/mL (high spike) of the semi-synthetic pS¹²⁹-α-syn standard. Twenty-five μL of each spiked or un-spiked sample were analyzed. The spike recovery rate was calculated as the ratio of the measured and the calculated concentration.

Dilution linearity. To test the dilution linearity, all biological samples were serially diluted 2-folds four times in Diluent 49 (MSD). The final volume of each sample was 25 μL. Final concentrations were measured and compared to the calculated concentrations based on the appropriate dilution factor.

Electrospray ionization-mass spectrometry (ESI-MS). The standard protein was dissolved at 1 mg/mL in diH₂O, buffer-exchanged into 20 mM ammonium acetate, pH 6.8, using 10-kDa MWCO Amicon centrifugal filters (Millipore Sigma, Burlington, MA), and diluted to 10 μM in the same buffer. The solution was electrosprayed using pulled nanoESI needles onto a Bruker 15T Solarix Fourier-transform ion cyclotron mass spectrometry system. The capillary voltage was set to 800V and the temperature to 180° C. The deflector plate was set to 160 V, the capillary exit to 100 V, the funnel voltage to 90 V, and the skimmer to 50 V. One hundred scans were collected to obtain the spectrum. The deconvolved spectrum was created using UniDec.¹⁹

SDS-PAGE and staining. 1, 0.5, 0.25, or 0.125 g of pS¹²⁹-α-syn and 1 μg of unphosphorylated α-syn were fractionated using Sure PAGE 4-20% gradient Bis-Tris gels (GenScript). Samples were prepared by mixing the protein solution with sample buffer (GenScript) and heated at 95° C. for 10 min. Gels were stained in 0.1% (w/v) Coomassie Brilliant Blue (Thermo Fisher Scientific) in 40% (v/v) methanol and 10% (v/v) acetic acid for 1 h at RT and then de-stained in the same solution excluding Coomassie Brilliant Blue. Silver-staining was performed using the SilverXpress Silver Staining Kit (Invitrogen) following the manufacturer's protocol.

Immunoblotting. Following SDS-PAGE fractionation, the proteins were transferred onto polyvinyl difluoride (PVDF) membranes (Thermo Fisher Scientific) for 1 h at 25 V on ice using XCell II Blot Modules (Invitrogen). Membranes were blocked in 5% non-fat dry milk in TBS-T for 1 h at RT. The membranes were probed with MJFR1 or EP1536Y at 1:1,000 dilution. The membranes then were washed and incubated with HRP-conjugated goat anti-rabbit antibody (Thermo Fisher Scientific) at 1:10,000 dilution in blocking solution for 1 h at RT, developed using SuperSignal West Pico PLUS Chemiluminescent Substrate (Life Technologies), and visualized using an Azure Biosystems c300 Gel Imager.

Data processing and statistical analysis. The sensitivity and dynamic range of each antibody combination were evaluated by determination of limit of blank (LoB), lower limit of detection (LLoD), lower limit of quantification (LLoQ), and upper limit of quantification (ULoQ). The applied definitions for the LoB, LLoD, LLOQ, and ULoQ are described in Table 1. Data were processed using MSD Discovery Workbench 4.0 software and Prism 9.4 (GraphPad, USA). Standard curves and sample concentrations were calculated using a four-parameter fit and plotted as mean #standard deviation of 4 replicates. Intra-assay coefficient of variation (CV) was calculated between standard concentrations measured on the same plate, whereas inter-assay CV was calculated in three independent experiments using different plates. Signal/background (S/B) and signal/noise (S/N) ratios were calculated as averages of three standard concentration points within the linear range of the assay for each antibody pair. Total percent error (TE %) was calculated as: ((calculated concentration−actual concentration)+2 SD)/actual concentration)×100. Relative percent error (RE %) was calculated as (calculated concentration−actual concentration)/actual concentration)×100 (see Supplementary Information).

TABLE 1
Definitions of assay parameters.²⁰

| | |
|---|---|
| LoB | Calculated concentration based on the mean zero calibrator |
| | signal + 1.645 × the standard deviation (SD) of the zero |
| | calibrator. |
| LLoD | Calculated concentration based on the signal + 1.645 × SD |
| | (lowest calibrator) above the LoB. |
| LLoQ | Calculated concentration based on the signal + 10 × SD (blank) |
| | above the zero calibrator. |
| ULoQ | Calibrator concentration at the upper limit of the linear range on |
| | a logarithmic scale. |

### Results

Evaluation of capture and detection antibody pairs and cross-reactivity with unphosphorylated α-syn

We chose to use the MSD ECLIA platform for development of a pS¹²⁹-α-syn assay as it offers high sensitivity detection at a relatively affordable price and has become widely used in academic institutions, the biotechnology industry, and pharmaceutical companies. The detection principle in this method is based on light emission from electrochemiluminescent labels conjugated to the detection antibody upon application of voltage to the printed electrodes on the back of the wells. The analyte is captured by biotinylated antibodies bound to a streptavidin-coated plate surface. Having made this choice, we tested different combinations of capture and detection antibodies for the degree of sensitivity and reproducibility of the measurements. A number of antibodies with variable sensitivity and specificity have been developed for specific recognition of pS¹²⁹-α-syn, some of which are commercially available.²¹ A recent study comparing several such antibodies found that the rabbit monoclonal antibody EP1536Y had the highest sensitivity and specificity for pS¹²⁹-α-syn,²² consistent with a previous study.²³ Some concerns about cross-reactivity of this antibody have been raised in a bioRxiv manuscript by Arlinghaus et al.²⁴ but were not reproduced in a recent, thorough characterization of six anti-pS¹²⁹-α-syn antibodies by Lashuel et al.²¹ Therefore, we decided to use this antibody either for capture or for detection in combination with an antibody that recognizes α-syn regardless of its phosphorylation status. Reports in the literature and our own experience suggested that mAb MJFR125 might be a good candidate for this task. In addition, we tested the anti-human q-synuclein antibody supplied with the commercial ECLIA kit sold by MSD.

We tested three different combinations of capture and detection antibodies (Table 2). In two combinations, EP1536Y was used either as a biotinylated capture antibody paired with Sulfo-tag anti-human α-synuclein (MSD) as the detection antibody or as a sulfonated detection antibody combined with a biotinylated MSD anti-human α-synuclein capture antibody. In the third combination, biotinylated MJFR1 was used for capture and Sulfo-tag EP1536Y for detection.

TABLE 2
Evaluation of antibody combinations for development of
the pS¹²⁹-α-synuclein electrochemiluminescence ELISA.

| | | | | | | Intra- | Inter- |
|---|---|---|---|---|---|---|---|
| | | | | | | assay | assay |
| Capture | Detection | LoB | LLoD | LLoQ | ULoQ | CV | CV |
| antibody | antibody | (pg/mL) | (pg/mL) | (pg/mL) | (pg/mL) | (%) | (%) |
| Biotin- | Anti- | 3.1 ± 0.6 | 5.8 ± 2.7 | 14.4 ± 10.0 | 66,167 | 3.9 | 7.2 |
| EP1536Y | human α- | | | | | | |
| (anti- | syn-Sulfo | | | | | | |
| pS¹²⁹-α- | tag | | | | | | |
| syn) | (MSD) | | | | | | |
| Biotin- | EP1536Y- | 10.7 ± 2.8 | 15.0 ± 7.5 | 75.1 ± 12.5 | 33,088 | 4.6 | 10.5 |
| anti- | Sulfo tag | | | | | | |
| human α- | | | | | | | |
| syn | | | | | | | |
| (MSD) | | | | | | | |
| Biotin- | EP1536Y- | 10.6 ± 10.1 | 31.9 ± 10.2 | 97.3 ± 29.7 | 33,088 | 7.2 | 28.3 |
| MJFR1 | Sulfo tag | | | | | | |
| (anti- | | | | | | | |
| human α- | | | | | | | |
| syn) | | | | | | | |

Mean ± SD are shown for LoB, LLoD and LLoQ.

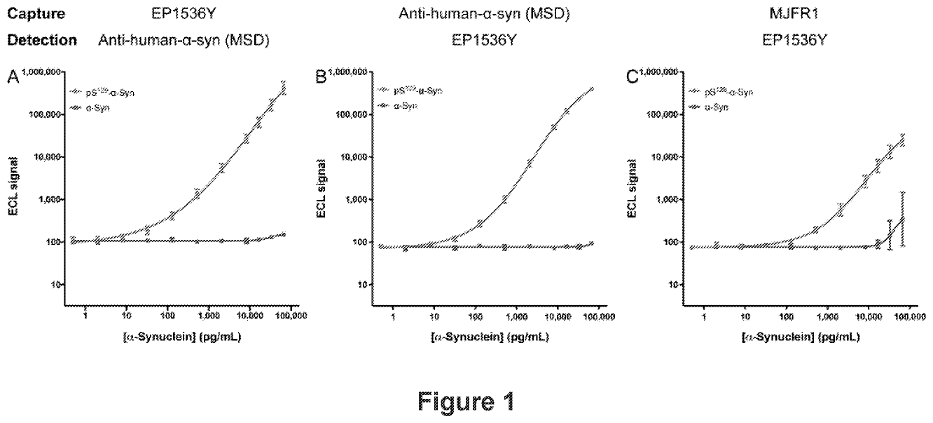

The sensitivity and reproducibility of each antibody combination were evaluated based on a standard curve generated using the semi-synthetic pS¹²⁹-α-syn at concentrations ranging from 0.5 to 66,167 μg/mL. Unphosphorylated α-syn standards were used to evaluate the specificity of the assay for the phosphorylated form. Each antibody combination was evaluated in at least five experiments performed in triplicate for each standard. Representative standard curves are shown in FIG. 1. Comparison of the sensitivity of the assays using EP1536Y and the anti-human α-synuclein (MSD) antibodies (Table 2) showed that when the former was used as capture and the latter for detection, the sensitivity was substantially higher (LLoD=5.8 μg/mL, LLoQ=14.4 pg/mL) than the reciprocal configuration (LLoD=15.0 μg/mL, LLoQ=75.1 μg/mL). The two configurations had comparable signal-to-baseline (S/B) and signal-to-noise (S/B) ratios (Supplementary Table 1). In comparison, when mAb MJFR1 was used for capture and EP1536Y for detection, the sensitivity was even lower-LLoD=31.9 pg/mL, LLoQ=97.3 μg/mL (Table 2) and the S/B and S/N values also were substantially lower than those of the two first antibody configurations. Therefore, we did not test the reciprocal configuration. All three configurations showed excellent specificity for pS¹²⁹-α-syn relative to its unphosphorylated form (FIG. 1). Of note, the most sensitive configuration, using biotin-EP1536Y for capture and MSD's anti-human α-synuclein antibody for detection, also had the best reproducibility compared to the other configurations. The intra- and inter-assay CV values were 3.9% and 7.2%, respectively, for this configuration, which also bad the widest dynamic range. Thus, this configuration was chosen for subsequent experiments, in which we sought to demonstrate the utility of the assay for analysis of biological samples. The calculated concentrations, TE % and RE % for the three antibody configurations in Table 1 are given in Supplementary Tables 2-4, respectively.

Determination of pS¹²⁹-α-Syn Levels in Biological Samples

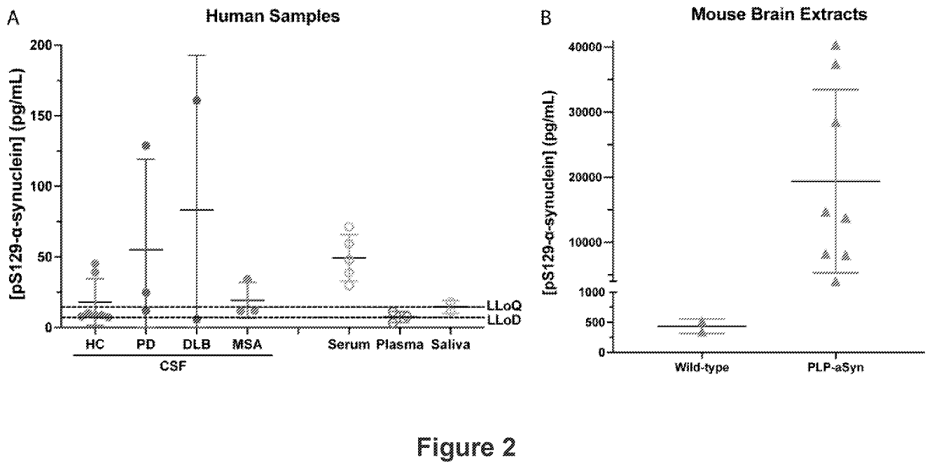

We determined the concentration of pS¹²⁹-α-syn in CSF samples from seven healthy control subjects as well as patients with synucleinopathies (3 PD, 2 DLB, 3 MSA). The pS¹²⁹-α-syn concentration levels were variable (FIG. 2A) and the small sample numbers did not allow meaningful comparison among the groups. In different samples of commercial, pooled, human serum we determined pS¹²⁹-α-syn levels to be 58±23 μg/mL whereas in human plasma the concentration was 7.4±3.3 μg/mL, suggesting that the anticoagulants in the plasma might interfere with the assay. Measurement of pS¹²⁹-α-syn concentrations in the saliva of two healthy control subjects yielded an average concentration of 14.5 #3.3 μg/mL (FIG. 2A). Most samples were above the LLoD yet many were below the LLOQ of the assay, likely reflecting the fact that except for the synucleinopathy CSF samples, all others were from healthy people, in which the levels of pS¹²⁹-α-syn are expected to be low. We also analyzed brain lysates from wild-type mice and PLP-α-syn mice, a transgenic model of multiple system atrophy in which α-syn is overexpressed under the PLP promoter,¹⁷ leading to its accumulation in oligodendrocytes.²⁶ The concentration of pS¹²⁹-α-syn in the soluble fraction of mouse brain extracts (5 μg of total protein) from the PLP-α-syn mice was 19,381±13,173 μg/mL, >50-fold higher than in brain extracts from wild-type mice (433±120 μg/mL, FIG. 2B). These findings are in agreement with the known accumulation of the phosphorylated protein in the brains of the MSA-model mice,²⁷ demonstrating the utility of the assay for mouse experiments. Finally, we isolated neuronal EVs (nEVs) and oligodendroglial EVs (oEVs) from the serum of healthy controls, patients with PD, and patients with MSA, as described previously,¹² and measured the pS¹²⁹-α-syn levels in these samples. The analysis showed concentrations between 1.5 and 248.9 μg/mL for nEVs and between 2.2 and 379.9 μg/mL for oEVs. Details will be published separately and therefore the data were not included in FIG. 2 and are mentioned here only for demonstration of feasibility.

### Dilution Linearity and Spike Recovery

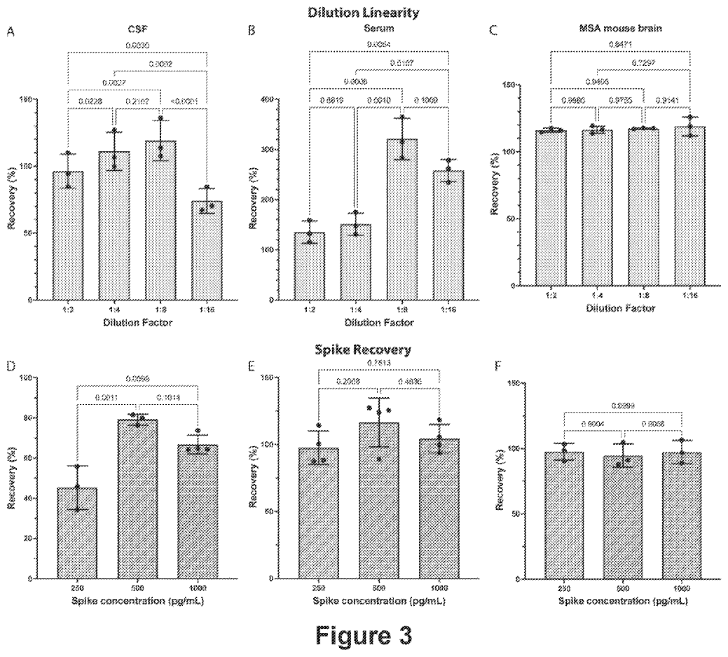

Next, we tested the dilution linearity and spike recovery in biological samples, different from those used in the previous experiments, including human CSF from a patient with DLB (undiluted concentration 113±14 μg/mL), pooled human serum (undiluted 39±9 μg/mL), and a PLP-α-syn mouse brain lysate (undiluted 14,265±457 μg/mL, FIG. 3C). Acceptable recovery rates were defined as 100±20%. The experiments showed reasonable linearity in the CSF up to 1:8 (FIG. 3A) and in the serum up to 1:4 dilution (FIG. 3B), likely reflecting the drop of the concentration at higher dilutions below the LLOQ. The dilution linearity was consistent for the PLP-α-syn mouse brain lysate samples (FIG. 3C) thanks to the high concentration of pS¹²⁹-α-syn in these samples, maintaining the concentration well above the LLOQ at all dilutions.

Spike recovery was tested in the same CSF, serum, and MSA mouse-brain extract samples at three concentrations-250, 500, and 1,000 μg/mL. These concentrations were chosen to cover the lower end of the dynamic range, in view of the low concentrations we detected in the human samples (FIG. 2). Good recovery was observed in both the serum (FIG. 3E) and mouse-brain extract (FIG. 3F) samples, whereas the recovery in CSF samples was low, particularly in the samples spiked with low-concentrations (FIG. 3D) suggesting that CSF components partially interfere with the signal and therefore the concentrations calculated in this medium might be an underrepresentation of the actual pS¹²⁹-α-syn concentration.

Evaluation of pS¹²⁹-α-Syn Standard Stability

During the course of the work described above, we observed on multiple occasions deterioration of the signal of the pS¹²⁹-α-syn standard within 1-2 weeks of storage, prompting a detailed investigation to identify the possible causes of this issue and potential solutions. We compared reconstitution of the protein in diH₂O or in TBS, pH 7.4, with or without pre-treatment with trifluoroacetic acid (TFA), filtered through 10- or 100-kDa MWCO filters to remove pre-formed aggregates, and used 1.0 mg/mL or lower concentrations of the standard protein for the stock solution. In all cases, the solution was aliquoted into single-use aliquots and stored at −80° C. until it was used in the assay. When this solution was used immediately after preparation, the ECLIA signal for the highest-concentration standard, 100 ng/mL, was between 5×10⁵ and 1.1×10⁶. This level of variability is similar to that reported by MSD for their total α-syn ECLIA kits. However, on multiple occasions, within 1-2 weeks of preparing the stock solution, the signal for this standard deteriorated by 10-20-fold, raising concern that the dynamic range of the assay would be too low for meaningful measurement and comparison among experiments would become difficult.

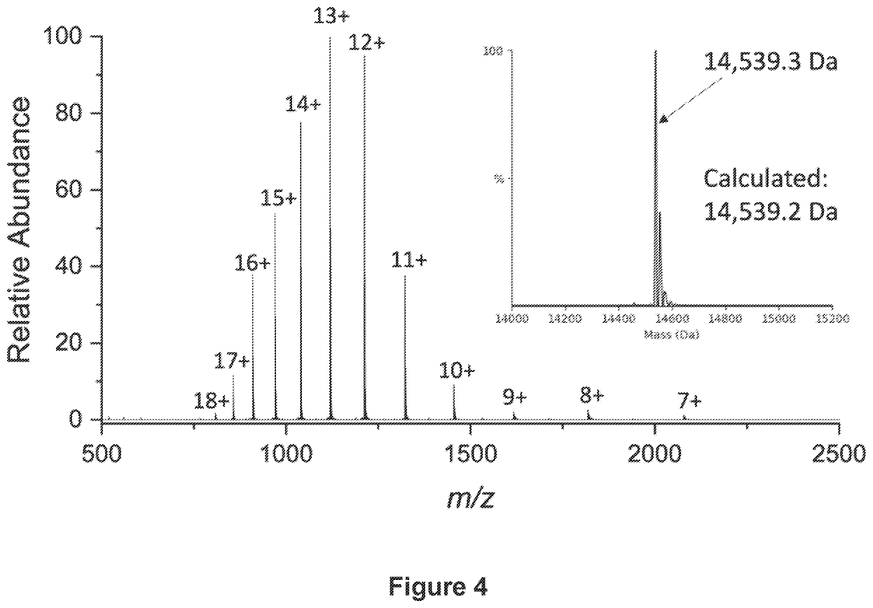

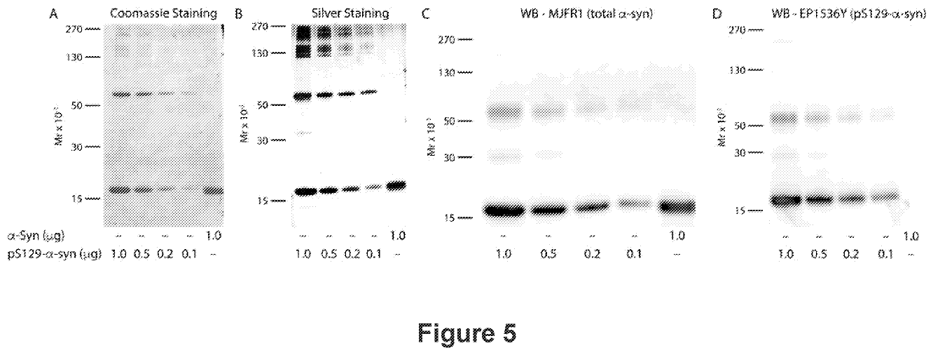

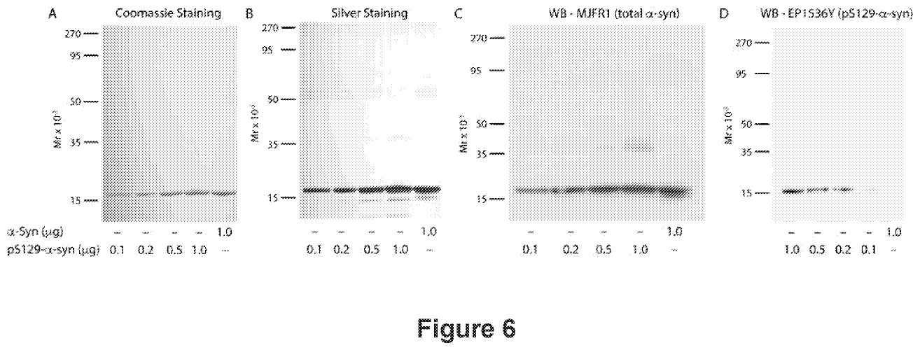

In view of the observed signal deterioration during the storage of the diluted and aliquoted protein standard, we tested whether it might have lost the phosphate group on Ser¹²⁹. Examination of the protein by ESI-MS revealed that it had the intact mass with the expected phosphorylation (FIG. 4), ruling out this option. Thus, we hypothesized next that the loss of signal could be due to oligomerization or aggregation of the protein during the preparation process, creating seeds that promoted further rapid aggregation, even when the protein is stored at −80° C. Indeed, under most of the conditions we used, we observed high-molecular weight bands of pS¹²⁹-α-syn using SDS-PAGE fractionation followed by Coomassie Blue staining (FIG. 5A), silver-staining (FIG. 5B), or western-blots probed with antibodies MJFR1 (FIG. 5C) or EP1536Y (FIG. 5D). Unphosphorylated α-syn was used as a control in these experiments and migrated as a monomer only (FIG. 5A-C). Changing the solution from diH₂O to TBS and filtering the stock solution did not resolve the issue. Reducing the concentration of the stock solution to 0.29 mg/mL, as recommended by Cariulo et al.¹¹ reduced the aggregation but signal loss was still observed if the TFA used for initial dissolution of the protein powder was not removed completely. Thus, following the protocol published by Cariulo et al.¹¹ precisely and ensuring that the TEA was removed completely, was necessary for preventing seed formation and allowing the protein to remain unaggregated for prolonged storage. Under these conditions, weekly repeated testing of the standard curve and positive control samples (MSA mouse-brain extract) yielded consistent data over one month without apparent signal loss. Analysis of the protein prepared in this manner using SDS-PAGE followed by Coomassie Blue staining (FIG. 6A), silver-staining (FIG. 6B), or western blots probed with MJFR1 (FIG. 6C) or EP1536Y (FIG. 6D) showed an absence of the previously observed high molecular weight bands.

### Discussion

High throughput measurement of disease-relevant post-translationally modified forms of α-syn with high sensitivity currently is an unmet need for clinical and biomarker studies. To address this need, we developed a novel assay based on the MSD ECLIA platform that detects and quantifies pS¹²⁹-α-syn at low pg/mL concentrations in various biological samples. The assay does not detect unphosphorylated forms of α-syn up to concentrations >10 ng/ml (FIG. 1). The specificity and high sensitivity of the assay are achieved thanks to the use of monoclonal antibody EP1536Y for capture of the analyte followed by detection by the Sulfo-Tag anti-α-syn antibody provided in MSD's total α-syn kit. Compared to the two other antibody combinations/configurations we attempted, this capture and detection combination provided not only the highest sensitivity, but also the highest dynamic linear range and best reproducibility. In agreement with this finding, other groups showed that the EP1536Y antibody is highly specific for pS¹²⁹-α-syn and could robustly detect it even in the presence of other post-translational modifications in close proximity to Ser¹²⁹ compared to other evaluated anti-pS¹²⁹-α-syn antibodies.^{14, 21, 22, 28} However, interference of other post-translational modifications and phosphorylation at other sites of α-syn were not assessed in our assay. Lashuel and colleagues reported that monoclonal antibody EP1536Y does detect α-syn phosphorylated at both Tyr125 and Ser¹²⁹ and does not detect pS¹²⁹-α-syn truncated after residues 133 or 135. In addition, the antibody showed reduced signal when tested for binding of α-syn fibril phosphorylated at Ser¹²⁹ and nitrated at Tyr125, Tyr133, and Tyr136 compared to fibrils of pS¹²⁹-α-syn itself.²¹ The contribution of these other post-translationally modified α-syn forms to the signal in the biological samples we tested here or to the levels of pS¹²⁹-α-syn measured in previous studies currently is not known.

When comparing the ECLIA platform described here with other published methods for quantifying pS¹²⁹-α-syn in biological fluids, it is important to note the lack of agreement in the data from different groups. For example, Wang et al. reported pS¹²⁹-α-syn concentrations of 58.1±20.2 and 79.2±23.2 μg/mL in the CSF of patients with PD or MSA using a bead-based Luminex assay,¹⁰ whereas Majbour et al. found substantially higher pS¹²⁹-α-syn concentration levels, 180.5-275 μg/mL in the CSF of healthy individuals and 206.8-296.3 μg/mL in the CSF of patients with PD determined by a sandwich ELISA.⁷ These assays used casein kinase II to phosphorylate recombinant α-syn, which was used as a standard, and Wang et al., diluted their samples by ¾, whereas Majbour et al. did not. The pS¹²⁹-α-syn concentrations we detected in CSF samples were mostly between 5-45 μg/mL (FIG. 2), somewhat lower than the findings of Wang et al. Only two samples, one from a patient with PD (129 μg/mL) and one from a patient with DLB (161 μg/mL) were closer to the levels reported by Majbour et al. In contrast, a study by Cariulo et al. did not detect pS¹²⁹ α-syn in pooled commercial CSF despite using a highly sensitive Singulex Erenna immunoassay with a LLoD of 0.15 μg/mL.¹¹ Using IP-MS/MS, Lashuel and co-workers also did not detect pS¹²⁹ α-syn in the CSF of patients with PD and healthy controls, highlighting the difficulty and inconsistency in measuring this α-syn species in CSF.²⁹ The differences in the measured pS¹²⁹-α-syn concentrations between these studies and our own can be attributed to several factors including, but not limited to, matrix effects, differences in the antibodies used, the use of recombinant versus semi-synthetic pS¹²⁹-α-syn for generating the standard curves, and most importantly the differences among the measurement platforms themselves.

To our knowledge, one previous study tested pS¹²⁹-α-syn in serum using a modified paired-surface plasma-wave biosensor and reported concentrations in the range 500 to 5,000 μg/mL in HC and 4,000 to 12,000 μg/mL in patients with PD.³⁰ However, the large differences between the technique used in that paper and the ECLIA used here makes comparison with our data difficult. Our data suggest that plasma components may interfere with the assay (FIG. 2). The plasma signal in our assay was an order of magnitude lower than the signal observed in serum samples. The two fluids differ mainly by the presence of anticoagulants in plasma, and possibly coagulation factors, e.g., fibrinogen, which may interfere with the assay. As both types of samples were commercial, pooled biofluids and not taken from the same persons, direct comparison was not possible. Previous studies by Foulds et al. in two separate cohorts found 200-600 ng/mL⁶ and 143.4±531.8 ng/mL pS¹²⁹-α-syn¹³ in control human plasma, several orders of magnitude above the concentrations we measured. However, these results also were over three orders of magnitude higher than the concentrations we found in serum, suggesting that assay differences likely were the main reason for these large discrepancies. In the study by Cariulo et al., pS¹²⁹ α-syn levels in the range 479.6-1223.2 μg/mL were found for clinically obtained and commercially pooled plasma samples.¹¹ The large discrepancy in values may be attributed to the differences in the assays themselves, batch variation, sample source, sample processing, and the antibody pairs used, as discussed above.

Notwithstanding the limitations discussed above, our results suggest that the pS¹²⁹-α-syn ECLIA is an attractive tool for measuring pS¹²⁹-α-syn in serum, saliva, potentially other biofluids, brain lysates, or other experimental systems, such as cell-cultures and animal models. Nevertheless, cross-validation of our assay by other groups, thorough validation of the used antibodies and strictly standardized sample preparation protocols are crucial for achieving reliable and reproducible results. We anticipate that future commercial kits will provide small aliquots of the standard that can be prepared freshly for a single use. Until such assays become available, researchers interested in using the assay are advised to follow carefully the protocol published by Cariulo et al.¹¹ to ensure that the semi-synthetic pS¹²⁹-α-syn, which currently is sold by Proteos only in 1-mg portions, is not lost due to aggregation shortly after its preparation.

### α-Synuclein Polypeptide Sequence: Accession P37840

See, e.g. Ueda et al., Proc. Natl. Acad. Sci. U.S.A. 90 (23), 11282-11286 (1993).

(SEQ ID NO: 1)
MDVFMKGLSKAKEGVVAAAEKTKQGVAEAAGKTKEGVLYVGSKTKEGV
VHGVATVAEKTKEQVTNVGGAVVTGVTAVAQKTVEGAGSIAAATGFVK
KDQLGKNEEGAPQEGILEDMPVDPDNEAYEMPSEEGYQDYEPEA

### REFERENCES DESCRIBING METHODS AND MATERIALS USEFUL IN ASPECTS OF THE INVENTION

1. Spillantini, M. G.; Crowther, R. A.; Jakes, R.; Hasegawa, M.; Goedert, M., α-Synuclein in filamentous inclusions of Lewy bodies from Parkinson's disease and dementia with Lewy bodies. Proc. Natl. Acad. Sci. U.S.A. 1998, 95 (11), 6469-6473.
2. Spillantini, M. G.; Schmidt, M. L.; Lee, V. M. Y.; Trojanowski, J. Q.; Jakes, R.; Goedert, M., α-Synuclein in Lewy bodies. Nature 1997, 388 (6645), 839-840.
3. Marti, M. J.; Tolosa, E.; Campdelacreu, J., Clinical overview of the synucleinopathies. Mov. Disord. 2003, 18 (S6), 21-27.
4. Anderson, J. P.; Walker, D. E.; Goldstein, J. M.; de Laat, R.; Banducci, K.; Caccavello, R. J.; Barbour, R.; Huang, J.; Kling, K.; Lee, M.; Diep, L.; Keim, P. S.; Shen, X.; Chataway, T.; Schlossmacher, M. G.; Seubert, P.; Schenk, D.; Sinha, S.; Gai, W. P.; Chilcote, T. J., Phosphorylation of Ser-129 is the dominant pathological modification of α-synuclein in familial and sporadic Lewy body disease. J. Biol. Chem. 2006, 281 (40), 29739-52.
5. Fujiwara, H.; Hasegawa, M.; Dohmae, N.; Kawashima, A.; Masliah, E.; Goldberg, M. S.; Shen, J.; Takio, K.; Iwatsubo, T., α-Synuclein is phosphorylated in synucleinopathy lesions. Nat. Cell Biol. 2002, 4 (2), 160-164.
6 Foulds, P. G.; Mitchell, J. D.; Parker, A.; Turner, R.; Green, G.; Diggle, P.; Hasegawa, M.; Taylor, M.; Mann, D.; Allsop, D., Phosphorylated α-synuclein can be detected in blood plasma and is potentially a useful biomarker for Parkinson's disease. FASEB J. 2011, 25 (12), 4127-37.
7. Majbour, N. K.; Vaikath, N. N.; Eusebi, P.; Chiasserini, D.; Ardah, M.; Varghese, S.; Haque, M. E.; Tokuda, T.; Auinger, P.; Calabresi, P.; Parnetti, L.; El-Agnaf, O. M. A., Longitudinal changes in CSF α-synuclein species reflect Parkinson's disease progression. Mov. Disord. 2016, 31 (10), 1535-1542.
8. MyBioSource Human Phosphorylated Alpha Synuclein ELISA Kit. https://www.mybiosource.com/psnca-human-elisa-kits/phosphorylated-alpha-synuclein/38716 (accessed 06. October 2022).
9. Majbour, N. K.; Vaikath, N. N.; van Dijk, K. D.; Ardah, M. T.; Varghese, S.; Vesterager, L. B.; Montezinho, L. P.; Poole, S.; Safieh-Garabedian, B.; Tokuda, T.; Teunissen, C. E.; Berendse, H. W.; van de Berg, W. D.; El-Agnaf, O. M., Oligomeric and phosphorylated α-synuclein as potential CSF biomarkers for Parkinson's disease. Mol. Neurodegener. 2016, 11, 7.
10. Wang, Y.; Shi, M.; Chung, K. A.; Zabetian, C. P.; Leverenz, J. B.; Berg, D.; Srulijes, K.; Trojanowski, J. Q.; Lee, V. M.; Siderowf, A. D.; Hurtig, H.; Litvan, I.; Schiess, M. C.; Peskind, E. R.; Masuda, M.; Hasegawa, M.; Lin, X.; Pan, C.; Galasko, D.; Goldstein, D. S.; Jensen, P. H.; Yang, H.; Cain, K. C.; Zhang, J., Phosphorylated α-synuclein in Parkinson's disease. Sci. Transl. Med. 2012, 4 (121), 121ra20.
11. Cariulo, C.; Martufi, P.; Verani, M.; Azzollini, L.; Bruni, G.; Weiss, A.; Deguire, S. M.; Lasbuel, H. A.; Scaricamazza, E.; Sancesario, G. M.; Schirinzi, T.; Mercuri, N. B.; Sancesario, G.; Caricasole, A.; Petricca, L., Phospho-S129 α-Synuclein Is Present in Human Plasma but Not in Cerebrospinal Fluid as Determined by an Ultrasensitive Immunoassay. Front. Neurosci. 2019, 13, 889.
12. Landeck, N.; Hall, H.; Ardah, M. T.; Majbour, N. K.; El-Agnaf, O. M.; Halliday, G.; Kirik, D., A novel multiplex assay for simultaneous quantification of total and S129 phosphorylated human α-synuclein. Mol. Neurodegener. 2016, 11 (1), 61.
13. Foulds, P. G.; Diggle, P.; Mitchell, J. D.; Parker, A.; Hasegawa, M.; Masuda-Suzukake, M.; Mann, D. M.; Allsop, D., A longitudinal study on α-synuclein in blood plasma as a biomarker for Parkinson's disease. Sci. Rep. 2013, 3, 2540.
14. Tian, C.; Liu, G.; Gao, L.; Soltys, D.; Pan, C.; Stewart, T.; Shi, M.; Xie, Z.; Liu, N.; Feng, T.; Zhang, J., Erythrocytic α-Synuclein as a potential biomarker for Parkinson's disease. Transl. Neurodegener. 2019, 8, 15.
15. Waxman, E. A.; Giasson, B. I., Specificity and Regulation of Casein Kinase-Mediated Phosphorylation of α-Synuclein. J. Neuropathol. Exp. Neurol. 2008, 67 (5), 402-416.
16. Fauvet. B.; Lashuel, H. A., Semisynthesis and Enzymatic Preparation of Post-translationally Modified α-Synuclein. In Protein Amyloid Aggregation: Methods and Protocols, Eliezer, D., Ed. Springer New York: New York, NY, 2016; pp 3-20.
17. Herrera-Vaquero, M.; Bouquio, D.; Kallab, M.; Biggs, K.; Nair, G.; Ochoa, J.; Heras-Garvin, A.; Heid, C.; Hadrovic, I.; Pocwe, W.; Wenning, G. K.; Klämer, F. G.; Schrader, T.; Bitan, G.; Stefanova, N., The molecular tweezer CLROI reduces aggregated, pathologic, and seeding-competent α-synuclein in experimental multiple system atrophy. Biochim Biophys Acta Mol Basis Dis 2019, 1865 (11), 165513.
18. Dutta, S.; Homung, S.; Kruayatidee, A.; Maina, K. N.; Del Rosario, I.; Paul, K. C.; Wong, D. Y.; Duarte Folle, A.; Markovic, D.; Palma, J. A.; Serrano, G. E.; Adler, C. H.; Perlman, S. L.; Poon, W. W.; Kang, U. J.; Alcalay, R. N.; Sklerov, M.; Gylys, K. H.; Kaufmann, H.; Fogel, B. L.; Bronstein, J. M.; Ritz, B.; Bitan, G., α-Synuclein in blood exosomes immunoprecipitated using neuronal and oligodendroglial markers distinguishes Parkinson's disease from multiple system atrophy. Acta Neuropathol. 2021, 142 (3), 495-511.
19. Marty, M. T.; Baldwin, A. J.; Marklund, E. G.; Hochberg, G. K.; Benesch, J. L.; Robinson, C. V., Bayesian deconvolution of mass and ion mobility spectra: from binary interactions to polydisperse ensembles. Anal Chem 2015, 87 (8), 4370-6.
20. Armbruster, D. A.; Pry, T., Limit of blank, limit of detection and limit of quantitation. Clin. Biochem. Rev. 2008, 29 Suppl 1 (Suppl 1), S49-52.
21. Lashuel, H. A.; Mahul-Mellier, A. L.; Novello, S.; Hegde, R. N.; Jasiqi, Y.; Altay, M. F.; Donzelli, S.; DeGuire, S. M.; Burai, R.; Magalhaes, P.; Chiki, A.; Ricci, J.; Boussouf, M.; Sadek, A.; Stoops, E.; Iseli, C.; Guex, N., Revisiting the specificity and ability of phospho-S129 antibodies to capture alpha-synuclein biochemical and pathological diversity. NPJ Parkinsons Dis 2022, 8 (1), 136.
22. Delic, V.; Chandra, S.; Abdelmotilib, H.; Maltbie, T.; Wang, S.; Kem, D.; Scott, H. J.; Underwood, R. N.; Liu, Z.; Volpicelli-Daley, L. A.; West, A. B., Sensitivity and specificity of phospho-Ser129 α-synuclein monoclonal antibodies. J. Comp. Neurol. 2018, 526 (12), 1978-1990.
23. Rutherford, N. J.; Brooks, M.; Giasson, B. I., Novel antibodies to phosphorylated α-synuclein serine 129 and NFL serine 473 demonstrate the close molecular homology of these epitopes. Acta Neuropathol. Commun. 2016, 4 (1), 80.
24. Arlinghaus, R.; Iba, M.; Masliah, E.; Cookson, M. R.; Landeck, N., Specific Detection of Endogenous S129 Phosphorylated α-Synuclein in Tissue Using Proximity Ligation Assay. bioRxiv 2021, 2021.09.28.461511.
25. Gray, M. T.; Munoz, D. G.; Gray, D. A.; Schlossmacher, M. G.; Woulfe, J. M., α-Synuclein in the appendiceal mucosa of neurologically intact subjects. Mov. Disord. 2014, 29 (8), 991-8.
26. Refolo, V.; Bez, F.; Polissidis, A.; Kuzdas-Wood, D.; Sturm, E.; Kamaratou, M.; Poewe, W.; Stefanis, L.; Angela Cenci, M.; Romero-Ramos, M.; Wenning, G. K.; Stefanova, N., Progressive striatonigral degeneration in a transgenic mouse model of multiple system atrophy: translational implications for interventional therapies. Acta Neuropathol. Commun. 2018, 6 (1), 2.
27. Stefanova, N.; Wenning, G. K., Animal models of multiple system atrophy. Clin Auton Res 2015, 25 (1), 9-17.
28. Rutherford, N. J.; Brooks, M.; Giasson, B. I., Novel antibodies to phosphorylated α-synuclein serine 129 and NFL serine 473 demonstrate the close molecular homology of these epitopes. Acta Neuropathol. Commun. 2016, 4 (1), 80.
29. Magalhaes, P.; Lashuel, H. A., Opportunities and challenges of α-synuclein as a potential biomarker for Parkinson's disease and other synucleinopathies. NPJ Parkinsons. Dis. 2022, 8 (1), 93.
30. Chen, W. R.; Chen, J. C.; Chang, S. Y.; Chao, C. T.; Wu, Y. R.; Chen, C. M.; Chou, C., Phosphorylated α-synuclein in diluted human serum as a biomarker for Parkinson's disease. Biomed. J. 2021.
All publications mentioned herein (e.g. Dutta et al., ACS Chem Neurosci. 2023 Apr. 5; 14 (7): 1238-1248; PCT publication WO 2020/146497 and those cited above) are incorporated herein by reference to disclose and describe aspects, methods and/or materials in connection with the cited publications (see also U.S. Patent Application Publication Numbers 20040096918, 20050255527, 20060019319, 20100316992, 20180037614, 20170176423, 20160273035, 20160370359, 20140315204, 20140367278, 20140299468, 20110275094, 20110177500, 20100221705 20100105145, 20180258496, 20150247854, 20150191798, 20110275094 and 20110189190).

### CONCLUSION

This concludes the description of embodiments of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

1. A method of measuring amounts of total α-synuclein and amounts of pS129-α-synuclein in a single sample comprising:
(a) combining the sample with:
a α-synuclein capture antibody and a α-synuclein detection antibody coupled to a detectable label; and
a pS129-α-synuclein capture antibody and a pS129-α-synuclein detection antibody coupled to a detectable label; wherein:
the α-synuclein capture antibody exhibits an affinity for α-synuclein greater than an affinity that the pS129-α-synuclein capture antibody exhibits for pS129-α-synuclein; and
amounts of the pS129-α-synuclein capture antibody and the amounts of the α-synuclein capture antibody are selected to form a concentration ratio that compensates for the different affinities;
(b) allowing the α-synuclein capture antibody and the α-synuclein detection antibody to bind α-synuclein
(c) allowing the pS129-α-synuclein capture antibody and the pS129-α-synuclein detection antibody to bind pS129-α-synuclein;
(c) observing the presence of the detectable labels; and
(d) correlating the observed detectable labels to concentrations of α-synuclein and pS129-α-synuclein in the sample;
such that amounts of total α-synuclein and amounts of pS129-α-synuclein in the single sample are measured.

2. The method of claim 1, wherein the concentration ratio is at least 1:8, 1:20 or 1:30.

3. The method of claim 1, wherein said correlating includes observing pS129-α-synuclein capture antibody binding to a recombinant pS129-α-synuclein polypeptide standard.

4. The method of claim 3, wherein:
the pS129-α-synuclein standard is selected to be in an unaggregated form;
the pS129-α-synuclein capture antibody and the recombinant pS129-α-synuclein polypeptide are incubated for at least 3 hours;
the pS129-α-synuclein capture antibody and the recombinant pS129-α-synuclein polypeptide are incubated at 4° C.; and/or
the method can quantify amounts of pS129-α-synuclein in in the sample in concentrations from about 1 to about 9 μg/mL.

5. The method of claim 3, wherein the sample is selectively obtained from:
an individual diagnosed with or suspected of having a synucleinopathy;
an individual who has suffered a traumatic brain injury or a spinal cord injury;
an individual being treated for a synucleinopathy; or
an individual being treated for a traumatic brain injury or a spinal cord injury.

6. The method of claim 1, wherein the method measures levels of pS129-α-synuclein present in brain-derived blood exosomes.

7. The method of claim 1, wherein the method comprises an electrochemiluminescent method for measuring pS129-α-synuclein in a sample comprising:
attaching a pS129-α-synuclein capture antibody to a matrix within a container, wherein the container comprises an electrode;
introducing a pS129-α-synuclein sample into the container so that pS129-α-synuclein binds the capture antibody;
introducing a pS129-α-synuclein detection antibody to the container so that the pS129-α-synuclein detection antibody binds pS129-α-synuclein, wherein the detection antibody comprises an electrochemiluminescent label;
applying electricity to the electrode so that light is generated by the electrochemiluminescent label;
measuring light emitted by the electrochemiluminescent label; and
correlating the measured light to a concentration of pS129-α-synuclein in the container.

8. The method of claim 6, wherein an anti pS129-α-synuclein antibody epitope comprises the amino acid sequence VDPDNE (SEQ ID NO: 2).

9. The method of claim 1, wherein:
the method comprises at least one step wherein α-synuclein detection antibody not bound to α-synuclein and pS129-α-synuclein detection antibodies not bound to pS129-α-synuclein are removed from the assay by a wash process; and/or
the sample volume is less than 20, 10 or 2 microliters.

10. A system for detecting amounts of α-synuclein and amounts of pS129-α-synuclein in a single sample comprising:
a pS129-α-synuclein polypeptide standard, wherein the pS129-α-synuclein standard is in an unaggregated form;
a pS129-α-synuclein capture antibody and a pS129-α-synuclein detection antibody coupled to an observable label;
a α-synuclein capture antibody and a α-synuclein detection antibody coupled to an observable label; wherein:
the α-synuclein capture antibody exhibits an affinity for α-synuclein greater than an affinity that the pS129-α-synuclein capture antibody exhibits for pS129-α-synuclein; and
amounts of the pS129-α-synuclein capture antibody and the amounts of the α-synuclein capture antibody are selected to form a concentration ratio that compensates for the different affinities.

11. The system of claim 10, wherein the concentration ratio is at least 1:20, 1:30 or 1:40.

12. The system of claim 10, wherein the system comprises a defined amount of a pS129-α-synuclein standard polypeptide useful to generate a standard curve in an electrochemiluminescent ELISA assay, and the pS129-α-synuclein standard/control is selected to remain in an unaggregated form following at least one month of storage at −80° C.

13. (canceled)

14. The system of claim 10, wherein:
the pS129-α-synuclein capture antibody and the recombinant pS129-α-synuclein polypeptide are incubated together for at least 3 hours; and/or
the pS129-α-synuclein capture antibody and the recombinant pS129-α-synuclein polypeptide are incubated at 4° C.

15. The system of claim 10, wherein an epitope bound by the pS129-α-synuclein antibody comprises the amino acid sequence VDPDNE (SEQ ID NO: 2).

16. The system of claim 10, wherein an epitope bound by the pS129-α-synuclein antibody comprises an epitope bound by antibody EP1536Y.

17. The system of claim 10, further comprising a container for performing an electrochemiluminescent ELISA assay.

18. The system of claim 17, wherein the container comprises an electrode material.

19. The system of claim 10, wherein the system further comprises unphosphorylated S129-α-synuclein polypeptide useful as a negative control.

20. (canceled)
